(19) United States
(12) Patent Application Publication
(10) Pub. No.: US 2002/0138120 A1
(43) Pub. Date: Sep. 26, 2002

(54) Radiation delivery method and apparatus

(76) Inventor: Colin Whitehurst, Cheshire (GB)

Correspondence Address: PILLSBURY WINTHROP, LLP, P.O. BOX 10500, MCLEAN, VA, 22102, US

(21) Appl. No.: 09/838,572

(22) Filed: Apr. 20, 2001

(30) Foreign Application Priority Data
Apr. 20, 2000 (GB) 0009901.0

(51) Int. Cl. A61N 5/06

(52) U.S. Cl. 607/88

(57) ABSTRACT

A fiber optic fabric 1 is described comprising optical fibers 2 woven with a fill material 3 where light is periodically emitted from the optical fibers. Such a fabric provides a flexible light source formable into garments for phototherapy or diagnosis.

### BACKGROUND OF THE INVENTION

The present invention relates to a method and apparatus for delivering electromagnetic radiation to an affected area of a patient, particularly but not exclusively in the treatment of skin conditions, particularly but not exclusively using photosensitizing drugs.

In known high-intensity phototherapy techniques, the affected area is treated using a narrow beam so as to concentrate the radiation onto the affected part. Where the affected part extends over a larger area, the treatment becomes very lengthy. In addition, the geometry of the treated areas can create serious problems in achieving uniform illumination.

Alternative devices have been proposed by which radiation may be applied at multiple points simultaneously. For example, U.S. Pat. No. 5,000,752 (Hoskin) discloses apparatus for treating port wine stains using a pad supporting a plurality of diamond-tipped needles, to which laser radiation is delivered through optical fibers. However, such apparatus is difficult to position correctly, requires the patient to be anaesthetized and is not suitable for treating large, non-planar areas.

The document U.S. Pat. No. 4,234,907 discloses a light-emitting fabric in which optical fibers are part of the weave. Light is emitted from the optical fibers through small scratches that pierce the outer coating. The fabric is said to be suitable for clothing, for example a suit for tanning in which UV light is emitted towards the user.

The document U.S. Pat. No. 4,907,132 discloses a light-emitting panel including one or more layers of woven fiber optic material having disruptions or bends at discrete locations along the length of the fibers to allow light to be emitted therefrom. The panel can be formed as a pad, belt, collar, blanket, strap or other such shape. The panel is said to be suitable for the treatment of jaundice in newborn babies.

The document U.S. Pat. No. 5,339,223 discloses a servo-control for a fibreoptic phototherapy pad of the type disclosed in U.S. Pat. No. 4,907,132.

One aim of the present invention is to allow phototherapeutic treatment of large, non-planar areas of the skin.

An alternative or additional aim of the present invention is to allow treatment of large areas without causing undue discomfort to the patient.

An alternative or additional aim of the present invention is to provide apparatus suitable for treatment of large areas of the skin with incoherent or non-laser light.

### STATEMENT OF THE INVENTION

According to one aspect of the present invention, there is provided a garment incorporating optical fibers connectable to a light source, and arranged such that light emitted from the fibers is directed towards a wearer.

According to another aspect of the present invention, there is provided a fiber optic fabric comprising a reflecting portion, a fiber optic portion including optical fibers and a diffusing portion, said fiber optic portion being positioned between said diffusing portion and said reflecting portion.

According to yet another aspect of the present invention, there is provided a fabric comprising a series of woven optical fibers, where the weaving causes any light transmitted through the optical fibers to escape from the fiber.

According to a further aspect of the invention there is provided fabric comprising a series of optical fibers treated so as to cause light transmitted through the optical fibers to substantially continuously escape along the length of the fiber.

According to a further aspect of the invention, there is provided use of a fabric or garment as described above for therapy or cosmetic treatment or photodynamic therapy.

Preferably, the garment or fabric includes a reflective layer positioned outside a layer of optical fibers, so as to reflect escaped light inwards.

Preferably, the garment or fabric includes a diffusing layer positioned inside the layer of optical fibers, so as to diffuse light emitted inwards.

Preferably, the garment or fabric includes means for coupling incoherent light from a light source into the optical fibers.

The garment or fabric may have a plurality of layers incorporating optical fibers.

### BRIEF DESCRIPTION OF THE DRAWINGS

The invention will now be described with reference to the following drawings in which:

FIG. 1 is a cross section through one form of fiber optic layer in accordance with the invention, showing the path of the light transmitted through an individual optical fiber;

FIG. 2 is an exploded perspective view of one form of material in accordance with the invention, incorporating a layer as shown in FIG. 1, showing connection of the optical fibers to a suitable light source;

FIG. 3 is an exploded perspective view of a second embodiment of the invention, showing a further form of material including layers comprising side emitting optical fibers; and

FIG. 4 is a schematic perspective view of a further embodiment of the invention, showing a plurality of fiber optic layers, as shown in FIG. 1, attached together to form a larger blanket.

### DESCRIPTION OF PREFERRED EMBODIMENTS

Light can be transmitted through optical fibers by total internal reflection; that is, any light rays traveling through the fiber and incident on the internal walls of the fiber at angles greater than a critical angle to the perpendicular (where the critical angle=sin⁻¹.(1/n) where n is the refractive index of the fiber medium) are reflected back into the optical fiber.

Weaving such optical fibers together with suitable fill material forms a flexible fiber optic fabric layer 1 having advantageous properties in the field of PDT.

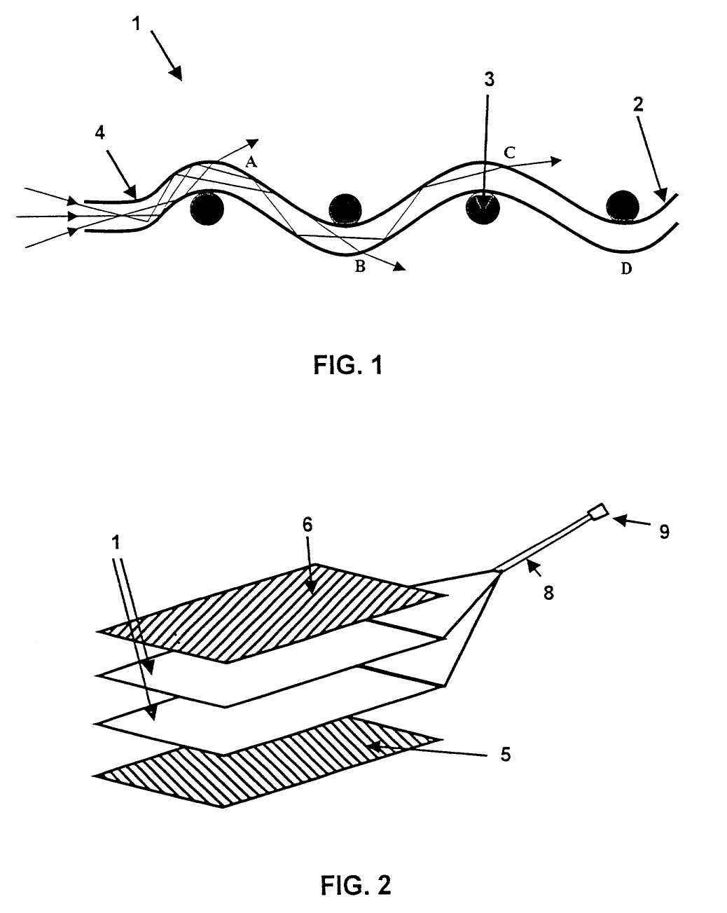

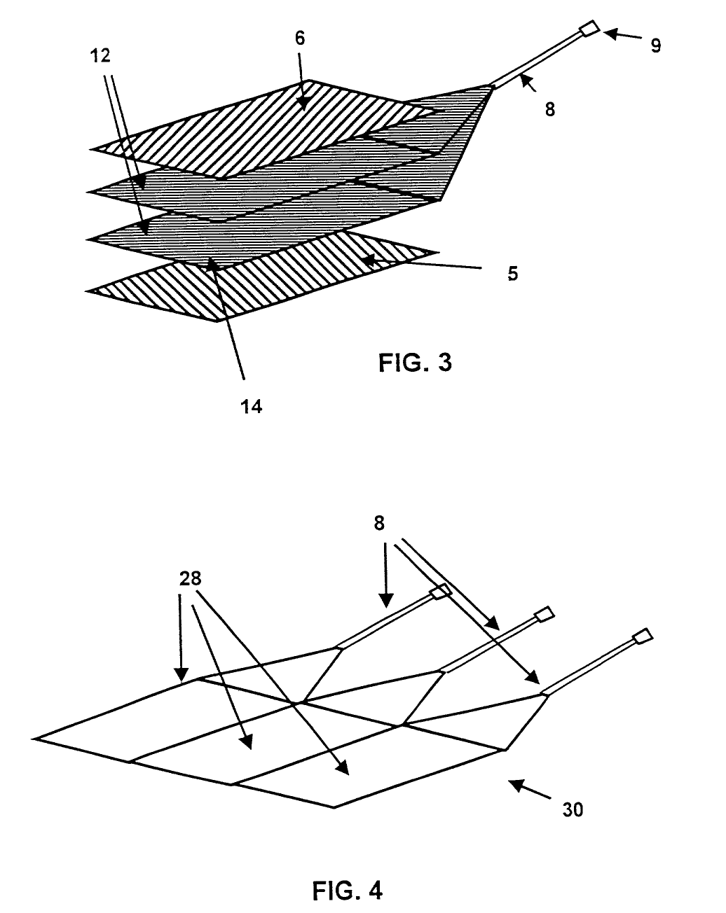

FIG. 1 illustrates a cross-section through such a flexible fabric layer 1 showing a section through one optical fiber 2 interwoven with several fill fibers 3.

As, in this case, the optical fiber 2 is bent around fill fibers 3, certain of the light rays transmitted through the optical fiber 2 will not be incident on the internal walls of the optical fiber 2 at angles greater than the critical angle, and as such will be refracted out of the optical fiber 2 and hence out of the layer of fabric 1.

As can be seen in FIG. 1, at points A, B, C and D, the light traveling through the optical fiber 2 is emitted from the side of the fiber 2 through cladding 4 on the optical fiber 2. It will be appreciated that when a large number of optical fibers 2 are woven in this way, the optical fiber fabric layer 1 formed will emit light in a generally uniform distribution across the fabric layer 1.

Referring to FIG. 2, two fiber optic fabric layers 1, as described above and shown in FIG. 1, are positioned between a diffusing layer 5 and a reflecting layer 6 to form a flexible multi-layered material suitable for use in PDT applications.

The diffusing layer 5 may be formed from a thin sheet of translucent plastic material that further diffuses the light emitted from the optical fibers 2. However, any suitable form of diffusing material may be used. The diffusing layer 5 will also act to protect the fiber optic fabric layer.

As the light emitted from the optical fiber fabric layers 1 is generally of a uniform distribution when the diffusing layer 5 is not present, it is not essential that a diffusing layer 5 be included.

A layer of transparent plastic material may be provided on the side of the optical fiber fabric layer 1 in place of the diffusing layer 5 to prevent damage on handling. The transparent plastic layer on the innermost surface of the optical fiber fabric layer 1 protects the optical fibers 2 in the fiber optical layer 1 when it is placed in contact with a patient undergoing PDT.

The reflecting layer 6 is a diffusely or specularly reflective layer provided on the surface of the optical fiber fabric layer 1 opposing the surface carrying the diffusing or protecting layer 5. The reflecting layer 6 acts to reflect light emitted towards the reflecting layer 6, from the fiber optic fabric layers 1, back towards the fiber optic fabric layers 1 and the diffusing layer 5.

The reflecting layer 6 may be formed from a Mylar (TM) diffuse reflecting sheet, a self-adhesive metallic vinyl sheet, such as DMX300 supplied by X-Film UK, Luton, Bedfordshire, or a thin aluminized or metallised plastic sheet.

Although the fiber optic fabric material shown in FIG. 2 includes two fiber optic fabric layers 1, it will be appreciated that any number of fiber optic fabric layers 1 may be used. Indeed, a single fiber optic fabric layer 1 may suffice.

The ends of the optical fibers 2 of the fiber optic layers 1 are brought together and formed into a fiber optic bundle 8. The ends are then terminated, polished and fixed, for example by adhesive, into a connector 9 for connection to a suitable PDT light source (not shown) emitting, in this case, non-laser or incoherent light. One example of a suitable light source is disclosed in UK Patent No 2272278 and is hereby incorporated in its entirety. The light source may have had the ultra-violet, infrared and non-active visible light removed, however, any suitable light source may be used. Depending on the number of optical fibers 2 involved, the diameter of the face of the bundle 8 may be in the region of 5 to 20 mm. This makes the material 1 particularly suited to connection to a non-laser light source, as these sources have an inherently large focused beam diameter of at least 5 mm.

Should a suitably small fiber optic bundle 8 be formed, it will be appreciated that the fiber optic fabric layers 1 described here may also be connected to a laser light source.

The optical fibers 2 are preferably formed from polymethyl methacrylate (PMMA) which has the appropriate transmission properties. However, it will be appreciated that any suitable glass material, such as E-glass, or plastic material having the appropriate light transmission properties may be used. The optical fibers are preferably clad but it will be appreciated that cladding is not essential.

Preferably, the diffusing layer 5 (if in use), the fiber optic layers 1 and the reflecting layer 6 are connected together at the edges by adhesive. However, it is not always necessary to attach the layers together at all. Furthermore, other suitable methods of attachment may be used such as stitching. Additionally, the layers need no be attached together at their edges but may be attached at any other suitable location.

In use, the material 1 is placed on a patient with the diffusing layer 5 closest to the patient and the light is transmitted from the PDT light source into the optical fibers 2 of the fiber optic fabric layers 1. The light is transmitted along the axis of the optical fibers 2 and periodically escapes from the top and bottom surface of the fiber optic fabric layer 1.

As shown in FIG. 1, the light escaping from the bottom surface, at points B and D, is transmitted toward the diffusing layer 5 and hence towards the patient. The light escaping from the top surface, at points A and C, is transmitted toward the reflecting layer 6 where it is reflected back toward the fiber optic layer 1, the diffusing layer 5 and hence the patient.

It will be appreciated that the fabric as shown in FIG. 2 is drawn in an exploded manner and that the diffusing layer 5, the optical fiber layers 1 and the reflecting layer 6 when in use appear as a single multi-layer flexible material. However, further layers of material may be positioned between the reflecting layer 6, the fiber optic layers 1 and the diffusing layer 5.

As the flexible material 1 is woven from elastically deformable fibers 2, a flexible light source is provided that could be manufactured to fit a wearer. For example, the fabric could be formed into the shape of a glove, a sleeve, a collar, or jacket or a large blanket for uniformly illuminating large non-planar areas of a wearer whilst at the same time maintaining wearer mobility and comfort.

Such a fabric or garment would provide a flexible PDT source for treatment of skin conditions or for use with photosensitizing drugs easily and comfortably wearable by any person. Fabrics or garments manufactured for such use would require the material to be arranged such that the diffusing layer 5 is positioned closest to the wearer, the fiber optic layer or layers 1 are positioned outwardly of the diffusing layer 5 relative to the wearer, and the reflecting layer 6 is positioned outwardly of the fiber optic layer or layers 1 relative to the wearer.

It will be appreciated that parameters such as flexibility, number of fiber optic layers and size of illuminating area need to be carefully chosen when the fabric is used for PDT. Different forms of treatment for different areas of the body will require different forms of fiber optic material 1. The flexibility of the material 1 depends on the diameter of the individual optical fibers, the material used for the diffusing layer, the thickness and rigidity of the diffusing layer and the ply or number of woven fiber optic fabric layers 1 employed.

For example, if a highly irregular or tightly curved area of the body is to be treated, a high degree of flexibility in the fiber optic material 1 is required. For these curved areas, the material 1 is formed using optical fibers 2 having a diameter of between 200 and 250 μm and between one and three fiber optic fabric layers 1. No diffusing layer 5 is included. Such a material 1 is highly flexible. For more planar areas of the body, where the material 1 does not have to be as flexible, more fiber optic fabric layers may be used, for example between three and six layers 1. Alternatively or additionally, optical fibers 2 having a larger diameter of around 300 μm may be used. A diffusing layer 5 may be employed.

The output intensity of the material 1 can be increased by increasing the optical power launched into the fiber optic bundle 8, by increasing the number of fiber optic fabric layers 1 or by a combination of increased power and increased number of layers 1.

The use of plastic optical fibers 2 can limit the optical power in the material 1. Increasing the optical power launched into the fiber optic bundle 8 by too large an amount may cause damage to the optical fibers 2 and may cause the connection between the fiber optic bundle 8 and the light source 10 to be damaged. For example melting of the fibers 2 may occur. The maximum power that can be used will depend on the ambient temperature, the degree of infrared filtration and the wavelength of the light used. In any case, the temperature of the fiber optic bundle 8 should not exceed 85° C.

Preferably, the light launched into the optical fiber has the ultra-violet and infrared portions filtered out. The light may also have any other non-active light removed. In this way, wastage of light and glare are reduced. This also prevents the fibers, and hence the material and the patient, from heating up substantially.

Furthermore, as the light is transmitted from the light source via light guides, the light source can be positioned remote from the patient and there is little electromagnetic (EM) radiation emitted transmitting the light via the fiber optic bundle 8 to the fiber optic fabric material in the vicinity of the patient. EM radiation can adversely affect heart pacemakers and other electronic equipment in use in hospitals. The fiber optic fabric material provides a flexible long-life PDT source that can be molded to uneven contours, like an item of clothing.

In general, therefore, to produce a uniform output, between two and four fiber optic fabric layers 1 should be used. Loss of flexibility and self-absorption of emitted light yields an upper limit of about six fiber optic fabric layers 1.

The size of the fabric panel to be used will also depend on the treatment being carried out. For example, a panel for wrapping around and treating the whole or partial circumference of a leg would require a surface approximately 15 cm by 60 cm. However, the dimensions of the active, light emitting area of the material 1 will vary depending on the size and number of lesions to be treated and the size of the patient's leg. A Velcro (TM) attachment would be provided for securing the material to the patient's body. Similarly, for treatment of an arm, a scaled down version of the above would be required.

For treatment of a torso, a scaled up version would be required. However, the material 1 could be formed into a garment such as a waistcoat for treatment of the front and back of the torso. This would advantageously secure the material 1 to the patient during treatment. Furthermore, it will be appreciated that other garments such as gloves, jackets or collars could be formed from the material 1 for treatment of appropriate parts of the body.

In a second embodiment of the invention, the fiber optic fabric layers 12 are formed from optical fibers 14 that are placed adjacent and in parallel to one another. These optical fibers 14 have surfaces that have been roughened, for example by chemical alteration such as etching or by rubbing their surfaces with glass paper. This changes the transmission properties of the optical fibers 14 and causes them to emit light from their surfaces along their length within the fiber optic fabric layer 1 in a manner similar to that described above with reference to the first embodiment of the invention. Fibers suitable for use in this way are supplied by Intelite, Inc., CA, USA. However, it will be appreciated that any suitable optical fibers may be used.

The optical fibers 14 are again brought together into one bundle 8 and connected to a suitable PDT light source for use in PDT as described above. Furthermore, the diffusing layer 5 and reflecting layer 6 may be included as described above.

In this way, a fiber optic layer for inclusion in a fiber optic fabric suitable for PDT may be formed without weaving optical fibers together with fill fibers.

The fabric formed using the fiber optic layers described in relation to the second embodiment of the invention, is used in the same manner described above in relation to the first embodiment of the invention.

In a further embodiment of the invention, as shown in FIG. 4, panels 28 including fiber optic layers 1, previously described above, are attached together to form a large blanket 30. Preferably, the layer or layers 1 of each panel 28 have their own fiber optic bundle 8 connected to a suitable PDT light source. In use, therefore, it is possible to activate only a portion of the whole blanket and treatment can be aimed only where required.

Similarly, fiber optic layers 1 or panel 28 could be incorporated into garments having non-active portions: for example, a waistcoat or jacket where the torso portion is formed from panels 28 or fiber optic layers 1 but the shoulder and/or sleeve portions are formed from non-fiber optic material. In this manner, the patient can easily wear the garment, whilst the panels 28 or fiber optic layers 1 are positioned correctly for treatment purposes.

A method of treatment for superficial cases of actinic/solar keratoses, Bowen's disease, superficial basal cell carcinoma, squamous cell carcinoma, intraepithelial carcinoma, mycosis fungoides, T-cell lymphoma, acne and seborrhoea, psoriasis, eczema, nevus sebaceous, viral infections such as herpes simplex, molluscum contagiosum, and warts (recalcitrant, verruca vulgaris or verruca plantaris), alopecia areata or hirsutism, using a fiber optic fabric material as detailed above, will now be described.

A cream or solution containing a photosensitizing drug such as 5-ALA is applied topically under medical supervision to the affected area of the skin of the patient, or administered intravenously or orally. For large areas, the patient may be immersed in a bath of solution. The affected area may then be covered for a period of 3 to 6 hours, or up to 24 hours if the treatment is to be continued the next day, to prevent removal of the drug and carrier, or activation by sunlight. The area is then uncovered and exposed to light from the light source, via the fiber optic fabric material, as described in any of the embodiments for a period of 15 to 30 minutes. The treatment may then be repeated as necessary, for a total of 1 to 3 treatments or more, depending on the severity and type of the condition. This method is particularly suitable for the treatment of patients with very large lesions or multiple lesions extending over a large area.

The light source may also be used for fluorescence detection (photodiagnosis).

The light source may also be used for cosmetic or partially cosmetic treatment with a photosensitizing drug for portwine stain removal and hair restoration/removal, and without a photosensitizing drug for skin rejuvenation, wrinkle removal or biostimulation (including wound healing).

The above embodiments are described purely by way of example and variants or alternatives may be envisaged which nevertheless fall within the spirit or scope of the present invention.

What is claimed is:

1. A wearable garment of woven material incorporating optical fibers connectable to a light source and woven such that light is emitted from the optical fibers towards a wearer.

2. A garment according to claim 1 in which said optical fibers form at least one layer of the garment.

3. A garment according to claim 2, further comprising a reflecting layer positioned outwardly of the fiber optic layer or layers relative to the wearer so as to reflect any outwardly emitted light back toward said wearer.

4. A garment according to claim 1, wherein the diameter of the optical fibers is between 200 and 250 μm.

5. A garment according to claim 2, further comprising a diffusing layer positioned inwardly of the fiber optic layer or layers relative to the wearer so as to diffuse said light directed toward the wearer.

6. A fiber optic fabric comprising a reflecting layer, one or more fiber optic layers each including optical fibers, and a diffusing layer, said fiber optic layer being positioned between said diffusing layer and said reflecting layer.

7. A fiber optic fabric according to claim 6, in which said fabric is formed in the shape of a wearable garment.

8. A fiber optic fabric according to claim 7, in which said diffusing layer is positioned inwardly of said fiber optic layer relative to a wearer of the garment.

9. A fiber optic fabric according to claim 7, in which said reflecting layer is positioned outwardly of said fiber optic portion relative to the wearer of the garment.

10. A fiber optic fabric according to claim 7, in which said reflecting portion acts so as to reflect any outwardly transmitted light inwardly toward said wearer.

11. A fiber optic fabric according to claim 7, in which said diffusing portion acts so as to diffuse any inwardly transmitted light directed toward said wearer.

12. A garment according to claim 1, in which the optical fibers are connected to a light source.

13. A fabric according to claim 6, in which the optical fibers are connected to a light-source.

14. A garment according to claim 13, in which said light source comprises an incoherent or non-laser light source.

15. A fabric according to claim 13, in which said light source comprises an incoherent or non-laser light source.

16. A garment according to claim 1, in which said optical fibers are formed from PMMA.

17. A fabric according to claim 6, in which said optical fibers are formed from PMMA.

18. A garment according to claim 1, in which said optical fibers are interwoven with fill fibers.

19. A fabric according to claim 6, in which said optical fibers are interwoven with fill fibers.

20. A garment according to claim 3, in which said reflecting layer is formed from Mylar (TM).

21. A fabric according to claim 6, in which said reflecting layer is formed from Mylar (TM).

22. A fabric including optical fibers treated so as to cause light transmitted through the optical fibers to escape substantially continuously along the length of the fibers.

23. A fabric according to claim 22, in which the treatment comprises chemical treatment of the surface of the fibers.

24. A fabric according to claim 22, in which the treatment comprises abrasion of the surface of the fibers.

25. A method of cosmetic treatment of a human or animal body, comprising: applying a photosensitizer to an area to be treated, and illuminating the area with light from a garment according to claim 1.

26. A method of cosmetic treatment of a human or animal body, comprising:
applying a photosensitizer to an area to be treated, and illuminating the area with light from a fabric according to claim 6 or claim 22.

27. A method of medical treatment of a human or animal body, comprising:
applying a photosensitizer to the area to be treated, and illuminating the area with light from a garment according to claim 1.

28. A method of medical treatment of a human or animal body, comprising:
applying a photosensitizer to the area to be treated, and illuminating the area with light from a fabric according to claim 6 or claim 22.

29. A method as claimed in any one of claims 25 to 28, for the treatment of one or more of: actinic/solar keratoses, Bowen's disease, superficial basal cell carcinoma, squamous cell carcinoma, intraepithelial carcinoma, mycosis fungoides, T-cell lymphoma, acne and seborrhoea, psoriasis, eczema, nevus sebaceous, viral infections such as herpes simplex, molluscum contagiosum, and warts (recalcitrant, verruca vulgaris or verruca plantaris), alopecia areata or hirsutism.

30. A method according to claim 29, wherein the condition is superficial.

31. A method according to any one of claims 25 to 28, wherein the photosensitiser includes or comprises PpIX.
